(19) United States
(12) Patent Application Publication
(10) Pub. No.: US 2020/0085043 A1
(43) Pub. Date: Mar. 19, 2020

(54) Antibacterial Formulations and Methods of Use

(71) Applicant: Prema Latha Bhat, Lutz, FL (US)

(72) Inventor: Prema Latha Bhat, Lutz, FL (US)

(21) Appl. No.: 16/131,956

(22) Filed: Sep. 14, 2018

(51) Int. Cl. A01N 37/02; A01N 37/36; A61K 8/36; A61K 8/365; A61K 8/9789; A61Q 11/00; A01N 65/22; A01N 65/44

(52) U.S. Cl. CPC A01N 37/02; A01N 37/36; A61K 8/36; A01N 65/44; A61K 8/9789; A61Q 11/00; A01N 65/22; A61K 8/365

(57) ABSTRACT

Provided herein are an alcohol-free anti-bacterial cleaner formulations. Generally, the anti-bacterial cleaner formulations contain vinegar and at least one organic acid in water. Also provided are the alcohol free antibacterial hard surface cleaner and brush cleaner formulations.

### BACKGROUND OF THE INVENTION

### Field of the Invention

The present invention relates generally to the field of anti-bacterial formulations. More specifically, the present invention relates to antibacterial formulations for disinfecting various surfaces including inanimate surfaces.

### Description of the Related Art

Antibacterial compositions and formulations include materials, which have the ability to disinfect. It is generally recognized that a disinfecting material greatly reduces or even eliminates bacteria existing on a surface. Compositions for oral hygiene and cleaning hard surfaces in the market use primarily antibiotics and antimicrobial active ingredients. For example, compositions based on quaternary ammonium compounds have been extensively described in the art for disinfecting purpose. While current products can provide temporary antibacterial disinfecting properties, they have not been well accepted by consumers looking for safer compositions.

The prior art is deficient in effective antibacterial disinfecting formulations which prevent infection by killing bacteria on inanimate surfaces. The present invention fulfills this longstanding need and desire in the art.

### SUMMARY OF THE INVENTION

The present invention is directed to an alcohol-free antibacterial cleaner. The antibacterial cleaner comprises water, vinegar in an amount of about 10% v/v to about 25% v/v and at least one organic acid in an amount of about 1% v/v to about 5% v/v. The present invention is directed to a related alcohol-free anti-bacterial cleaner further comprising at least one natural extract in an amount of about 0.25% v/v to about 20% v/v.

The present invention also is directed to an alcohol-free antibacterial hard surface cleaner. The antibacterial hard surface cleaner comprises water, a vinegar in an amount of about 25% v/v, at least two organic acids each in an amount of about 4% v/v, and at least one natural extract in an amount of about 0.5% v/v.

The present invention is directed further to an alcohol-free anti-bacterial brush cleaner. The alcohol-free anti-bacterial brush cleaner comprises water; vinegar in an amount from about 10%; at least one organic acid in an amount from about 1% v/v to about 5% v/v; and at least one natural extract in an amount of about 0.25% v/v.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1B show the bacterial count in the lauria broth plates without (FIG. 1A) and with (FIG. 1B) using the mouth wash #2 of the present invention, analyzed 30 min after use.

FIGS. 2A-2C show the results of the comparative bacterial count in the lauria broth plates in the control (FIG. 2A), after using mouthwash#2 (FIG. 2B) and after using commercially available mouthwash (LISTERINE containing 21% alcohol) after overnight sleep (FIG. 2C).

FIGS. 3A-3B show the bacterial count in the lauria broth plates before (FIG. 3A) and after (FIG. 3B) using toothbrush wash of present invention.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “essential oil” refers to a concentrated, hydrophobic liquid containing a volatile aromatic compound(s) from a plant that is produced by distillation.

As used herein, the term “chewing gum” refers to include not only chewing gums, but also any oral product which can be chewed, e.g., bubble gum, dragees, and the like.

As used herein, “hard surfaces”, typically refers to floors, walls, tiles, windows, floors, glass, bathroom surfaces, kitchen surfaces, dishes, appliances as well as fabrics, clothes, carpets and the like.

As used herein, “brush”, typically refers to tooth brush, hair brush, make-up brush and the like.

In one embodiment of the present invention, there is provided an alcohol-free anti-bacterial cleaner, comprising: water; vinegar in an amount of about 10% v/v to about 25% v/v; and at least one organic acid in an amount of about 1% v/v to about 5% v/v. Further to this embodiment, the alcohol-free anti-bacterial cleaner comprises at least one natural extract in an amount of about 0.25% v/v to about 20% v/v. Representative examples of a natural extract include, but are not limited to, neem extract, ginger extract, rosemary, lemon grass extract, mint, peppermint, mango leaves extract, lime extract, Hibiscus flower extract, green tea leaf extract, or fennel seed extract, or a combination thereof.

In both embodiments, representative examples of vinegar include, but are not limited to, 5% acetic acid, 10% acetic acid, balsamic vinegar or apple cider vinegar. In one aspect of both embodiments the vinegar may be present in an amount of about 10% v/v. In another aspect, the vinegar may be present in an amount of about 25% v/v.

Also in both embodiments, representative examples of a useful organic acid include, but are not limited to, citric acid, or propionic acid, or a combination thereof. In one aspect the organic acid may be present in an amount of about 1% v/v. In another aspect, the organic acid may be present in in an amount of about 4% v/v. Generally, in all embodiments and aspects thereof the alcohol-free anti-bacterial cleaner is effective to clean a hard surface, a toothbrush, a brush or a make-up brush.

In another embodiment of the present invention, there is provided an alcohol-free anti-bacterial hard surface cleaner comprising water; vinegar in an amount of about 25% v/v; at least two organic acids each in an amount of about 4% v/v; and at least one natural extract in an amount of about 0.5% v/v. In this embodiment, the hard surface may be floors, walls, tiles, windows, floors, glass, bathroom surfaces, kitchen surfaces, dishes, appliances as well as fabrics, clothes, carpets and the like. Also in this embodiment representative examples of a useful organic acid include, but are not limited to, citric acid and propionic acid. In addition representative examples of vinegar and natural extract are as described supra.

In yet another embodiment, there is provided an alcohol-free anti-bacterial brush cleaner comprising: water; vinegar in an amount from about 10%; at least one organic acids in an amount from about 1% v/v to about 5% v/v; and at least one natural extract in an amount of about 0.25% v/v. In this embodiment, the brush may be a hair brush, a tooth brush, a make-up brush and the like. Also in this embodiment representative examples of vinegar, organic acid and natural extract are as described supra.

In yet another embodiment of the present invention, there is provided an antibacterial and antioxidant oral care formulation comprising water, a vinegar in an amount of about 1% v/v to about 25% v/v; at least one natural extract in an amount of about 0.05% v/v to about 20% v/v; and a sodium bicarbonate in an amount of about 10% w/v. Sodium bicarbonate is used as a buffering agent to adjust pH to adjust pH to 6.8-7.0.

Further to this embodiment, the formulation comprises at least organic acid in an amount of about 0.25% v/v to about 2% v/v. Representative examples of a useful organic acid include but are not limited to citric acid, or propionic acid, or a combination thereof. In another further embodiment the formulation comprises at least one flavoring agent in an amount of about 0.05% v/v to about 0.25% v/v. Representative examples of a flavoring agent include but are not limited to lemon grass extract, mint, fennel seed extract, peppermint, rosemary or ginger extract, or a combination thereof. In yet another further embodiment the formulation comprises an oil in an amount about 5% to 25% v/v. Representative examples of useful oil include but are not limited to coconut oil, olive oil, tea tree essential oil, peppermint essential oil, or clove essential oil or a combination thereof. In yet another further embodiment the formulation comprises a polyhydric alcohol sugar in an amount of about 2% w/v. Representative examples of a polyhydric alcohol sugar include but are not limited to ethylene glycol, propylene glycol, 1,3-butylene glycol, polyethylene glycol, glycerine, xylitol, maltitol, lactitol, or sorbitol, or combination thereof.

In all embodiments representative examples of vinegar include, but are not limited to, 5% acetic acid, 10% acetic acid, balsamic vinegar or apple cider vinegar. Representative examples of a natural extract include, but are not limited to, neem extract, ginger extract, rosemary, lemon grass extract, mint, peppermint, mango leaves extract, lime extract, Hibiscus flower extract, green tea leaf extract, or fennel seed extract, or a combination thereof.

In one aspect of these embodiments, the antibacterial oral and antioxidant care formulation may be a mouthwash comprising the water, the vinegar in an amount of about 1% to about 5% v/v; at least one organic acid in an amount of about 0.5% v/v; at least two natural extracts each in an amount of about 1%; at least one flavoring agent in an amount of about 0.1% v/v and the sodium bicarbonate in an amount of about 10% w/v. Sodium bicarbonate is used as a buffering agent to adjust pH to adjust pH to 6.8-7.0.

In another aspect of the embodiment, the antibacterial and antioxidant oral care formulation may be a mouthwash comprising the water; vinegar in an amount of about 10% to about 25% v/v; at least two organic acids each in an amount of about 0.5% v/v to about 2% v/v; at least one natural extract in an amount of about 0.05% v/v to about 0.1% v/v; at least one flavoring agent in an amount of about 0.05% to about 0.2% v/v and sodium bicarbonate in an amount of about 10% w/v as a buffering agent to adjust pH to adjust pH to 6.8-7.0.

In yet another aspect of the embodiment, the antibacterial and antioxidant oral care formulation may be a mouthwash comprising water; the vinegar in an amount of about 5% v/v; at least one organic acid in an amount of about 0.5% v/v; at least two natural extract each in an amount of about 0.5% v/v to about 20% v/v; at least one flavoring agent in an amount of about 0.25% v/v to about 0.5% v/v and sodium bicarbonate in an amount of about 10% w/v. Sodium bicarbonate is used as a buffering agent to adjust pH to adjust pH to 6.8-7.0.

In yet another aspect of the embodiment, the antibacterial and antioxidant oral care formulation may be a mouthwash comprising water; the vinegar in an amount of about 5% v/v; at least one organic acid in an amount of about 0.5% v/v; at least two natural extract each in an amount of about 1% v/v to about 20% v/v; and sodium bicarbonate in an amount of about 10% w/v. Sodium bicarbonate is used as a buffering agent to adjust pH to adjust pH to 6.8-7.0.

In yet another embodiment of the present invention, there is provided an antibacterial and antioxidant toothpaste comprising water; a vinegar in an amount of about 1% v/v; a mango leaves extract in an amount of about 1% v/v; at least one oil in an amount of about 5% v/v to 25% v/v; a polyhydric alcohol in an amount of about 2% w/v; a flavoring agent in an amount of about 0.25% v/v; and a sodium bicarbonate in an amount of about 10% w/v. The formulation is buffered to pH 6.8-7.0. Representative examples of flavoring agents, vinegars, oils, and polyhydric alcohol sugars are as described supra.

In yet another embodiment of the present invention, there is provided an antibacterial chewing gum comprising a mango leaves extract in an amount of about 1% by weight of gum; a polyhydric alcohol in an amount of about 2% by weight of gum; a flavoring agent in an amount from about 0.25% by weight of gum; and a sodium bicarbonate in an amount of about 10% by weight of gum. In this embodiment, the chewing gum is in stick or a tablet form. Representative examples of flavoring agents and polyhydric alcohol sugars are described supra.

The gum base may be of any conventional nature known in the art. For example the gum base may be of natural or synthetic origin from a commercial source. Natural gum bases include, e.g., chicle, jelutong-, lechi de caspi-, soh-, siak-, katiau-, sorwa-, balata-, pendare-, malaya-, and peach gums, natural cautchouc and natural resins such as dammar and mastix. Synthetic gum bases are a mixture of elastomers (polymers, masticating substances), plasticizer (resin, elastomers, solvent, hydrophobic resin), filler (texturizer, water-insoluble adjuvant), softener (fat), emulsifier, wax, antioxidant, and anti-tacking agents (vinyl polymer, hydrophilic resin).

In yet another embodiment of the present invention, there is provided a sprayable breath freshener comprising: the water, the vinegar in an amount of about 1%; mango leaves extract in an amount of about 1% v/v; a polyhydric alcohol in an amount of about 2% w/v; at least one flavoring agent in an amount of about 0.25% v/v; and sodium bicarbonate in an amount of about 10% w/v. Sodium bicarbonate is used as a buffering agent to adjust pH to adjust pH to 6.8-7.0. Representative examples of vinegars, flavoring agents, and polyhydric alcohol sugars are as described supra.

Provided herein are oral antibacterial and antioxidant formulations effective to prevent or reduce the formation of bacteria on teeth, gums and disinfect hard surfaces. The oral formulations of the present invention have an extraordinary lingering effect as a barrier and are fully safe and nontoxic. The oral formulations of the present invention do not perform as an antibiotic by eliminating most of the normal oral microbial flora or by generating resistant strains, but help to prevent biofilm formation and, therefore, the formation of dental plaque and tartar, halitosis, periodontal diseases and to provide an antioxidant wash to oral muscosal surfaces. The oral formulations of the present invention provide natural antioxidants to oral surface.

The oral antibacterial and antioxidant formulations of the present invention may be used one or more times per day to prevent the formation of dental biofilm, to reduce or prevent bad breath due to putrefaction of food waste, to prevent tooth decay and periodontal disease and, therefore, to prevent daily bacteremia and systemic microbial conditions. Thus, methods of improving oral hygiene, such as, preventing or reducing the occurrence of biofilms or bacteria on the teeth also are provided.

The oral antibacterial and antioxidant formulations of the present formulation provide a non-toxic formulation being substantially or almost completely safe and effective and useful for anyone regardless of his or her clinical, medical, psychosocial, and economic situation. The oral antibacterial and antioxidant formulations include mouthwashes, toothpastes and breath fresheners.

Also provided are antibacterial formulations or antibacterial cleaners effective for disinfecting inanimate surfaces such as oral care products, cosmetic products, hard surfaces, such as walls, tiles, floors, glass, bathroom surfaces, kitchen surfaces, dishes, as well as fabrics, clothes, carpets and the like. The components of the formulations or cleaners comprise organic acids or natural extracts or a combination thereof and provide a non-toxic, safe and effective composition.

The formulations or cleaners of the present invention also have analgesic properties with many uses, for example, but not limited to, asepsis, dermal revitalization, topical analgesia and decreased skin-bleeding. Thus, the formulations or cleaners of the present invention are useful to treat, to alleviate or to provide analgesia for, but not limited to, sunburn, mild to moderate skin burns, dermal ulcers, abrasions, wounds, skin blemishes, and skin lacerations with or without loss of blood. Thus, methods of alleviating or treating a condition of the skin also are provided.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Preparation of Mouthwash #1

The mouthwash, hereinafter referred to as mouthwash #1 (MW1) was prepared by mixing Balsamic vinegar, citric acid, mango leaves extract; Hibiscus leaves extract, and 1M sodium bicarbonate in water. The pH of the mouth wash 1 was adjusted from about 6.5 to about 7 using sodium bicarbonate. The mouth wash 1 optionally includes flavoring agents such as lemon grass extract, mint extract, fennel seed extract or a combination thereof and coloring agents. Balsamic vinegar can be substituted with 5% acetic acid or apple cider vinegar. A representative formulation containing exemplary amounts of the components in water is shown in Table 1 below.

TABLE 1

| Components of MW#1 | Amount in 100 mL of water |
|---|---|
| Balsamic Vinegar | 1-5 |
| Citric acid | 0.5 mL |
| Mango leaves extract | 1 mL |
| Hibiscus flower extract | 1 mL |
| Lemon grass extract (optional) | 100 μL |
| Mint extract (optional) | 100 μL |
| Fennel seed extract (optional) | 100 μL |
| 1M Sodium Bicarbonate | 8-10 ml |

### EXAMPLE 2

### Antibacterial Effect of Mouthwash #1

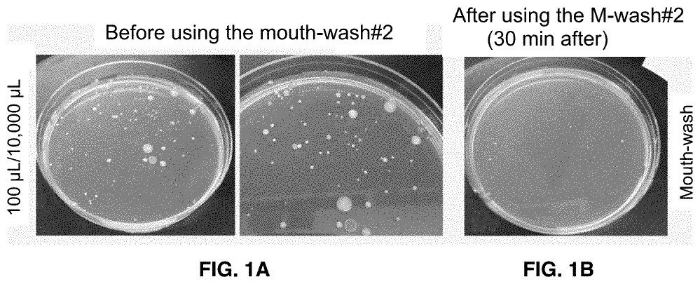

Water without the ingredients was used as a control. The mouthwash #1 shown in Table 1 and a control solution were tested on the same person one week apart without changing the diet or brushing (brush or toothpaste). The test person was asked to rinse the mouth thoroughly after brushing with regular brush either with control (25 mL, FIG. 1A) or with a mouthwash 1 (FIG. 1B) of the present invention (25 mL). Nothing was eaten after brushing and next morning the test person was given 10 ml of water to rinse thoroughly. The known amount of rinsed water was immediately cultured and tested for bacterial counts. Specified amounts of the wash was plated on lauria broth (lb) media plates and incubated overnight at 37° C. The pH of mouth samples was 6.5-7.0 for control as well as for the mouth wash 1. The colonies were then counted (FIGS. 1A-1B). Table 2 shows the bacterial count in 10 μl/10 ml of mouth rinse.

TABLE 2

| | | Bacterial count after |
|---|---|---|
| SN | Bacterial count Control | treatment with MW1 |
| 1 | 220 | 114 |
| 2 | 231 | 99 |
| 3 | 240 | 89 |

### P Value and Statistical Significance

The two tailed P value equals 0.0002. By conventional criteria, this difference is considered to be extremely statistically significant. Average bacterial count in control was 230 with a standard deviation of 10 and standard error of 5.7. Average bacterial count in samples after use of the mouth wash 1(MW1) was 100, with standard deviation of 12 and standard error of 7.2. The mean of Group One minus Group Two equals 129.67. 95% confidence interval of this difference was from 103.89 to 155.45.

### EXAMPLE 3

Preparation of Mouthwash #2 with Propionic Acid

The mouthwash hereinafter referred to as mouthwash #2 (MW2) was prepared by mixing 5% acetic acid, 100% propionic acid, 100% citric acid, neem extract, ginger extract, rosemary extract, lemon grass extract, mint extract, fennel seed extract and 1M sodium bicarbonate in water. The pH of the mouthwash 2 was adjusted from about 6.5 to about 7 using sodium bicarbonate. The neem extract, ginger extract, rosemary extract, lemon grass extract, mint extract or fennel seed extract can be used alone or in various/different combinations. A representative formulation containing exemplary amounts of the components in water is shown in Table 3 below.

TABLE 3

| Components of MW#2 | Amount in 50 mL of water |
|---|---|
| Acetic acid | 5-12.5 mL |
| Citric acid | 125 μL |
| Propionic acid | 0.5-1 mL |
| Neem extract | 25-100 μL |
| Ginger extract | 25-100 μL |
| Rosemary extract | 25-100 μL |
| Lemon grass extract | 25-100 μL |
| Mint extract | 25-100 μL |
| Fennel seed extract | 25-100 μL |
| 1M Sodium Bicarbonate | 8-10 mL |

### EXAMPLE 4

### Antibacterial Effect of Mouthwash #2

The mouthwash #2 (MW2) shown in Table 3 and a control were tested on the same person one week apart without changing the diet or brushing (brush or toothpaste). The test person was asked to rinse the mouth thoroughly after brushing with regular brush either with control (FIG. 1A) or with MW2 (FIG. 1B). After 30 minutes, the test person was given 10 ml of water to rinse thoroughly. A known amount of rinsed water was immediately cultured and tested for bacterial counts. Specified amounts of the wash was plated on lauria broth (lb) media plates and incubated overnight at 37° C. The pH of mouth samples was 6.5-7.0 for both the control as well as for the mouthwash 2. The colonies were then counted (FIGS. 1A-1B). Table 4 shows the bacterial count in 100 μl/10 ml of mouth rinse.

TABLE 4

| | | Bacterial count after |
|---|---|---|
| SN | Bacterial count Control | treatment with MW2 |
| 1 | 110 | 15 |
| 2 | 98 | 11 |
| 3 | 121 | 9 |

### P Value and Statistical Significance

The two tailed P value equals 0.0001. By conventional criteria, this difference is considered to be extremely statistically significant. Average bacterial count in control was 109 with a standard deviation of 11.5 and a standard error of 6.6. Average bacterial count in samples after use of the MW2 was 12 with a standard deviation of 3 a standard error of 1.7.

### EXAMPLE 5

### Antibacterial Effect of Mouthwash #2 vs LISTERINE

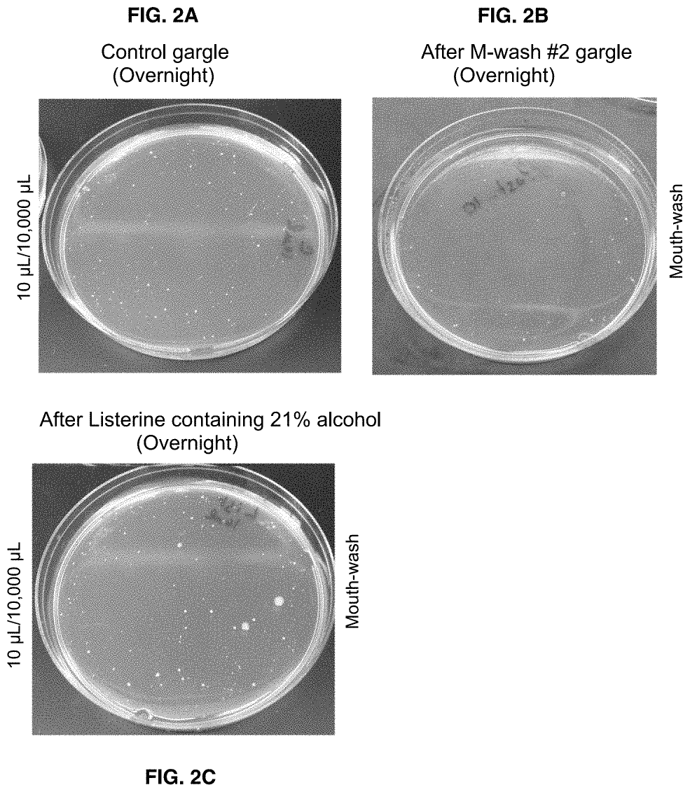

After brushing with regular toothbrush, a test subject gargled with either water (control, FIG. 2A) or the mouthwash #2 (MW2, FIG. 2B) shown in Table 3 or LISTERINE, a product of Johnson & Johnson. Nothing was eaten and next morning the subject gargled with 10 ml of water and a known amount of this was immediately cultured on lauria broth plates, colonies were counted after overnight incubation at 37° C. Table 5 shows the bacterial count in 10 μl/10 ml of rinsed control, mouthwash 2 and LISTERINE (FIG. 2C).

TABLE 5

| | | Bacterial count after | |
|---|---|---|---|
| SN | Bacterial count Control | treatment with MW2 | LISTERINE |
| 1 | 220 | 112 | 181 |
| 2 | 231 | 106 | 173 |
| 3 | 240 | 98 | 164 |

### P Value and Statistical Significance

The two tailed P value equals 0.0001 in case of control and MW2 and 0.0016 in case of control and LISTERINE. By conventional criteria, this difference is considered to be extremely statistically significant. Average bacterial count in control was 230 with a standard deviation of 10 and standard error of 5.7. Average bacterial count in samples after use of mouthwash #2 (MW2) was 105 with a standard deviation of 7 and standard error of 4. Average bacterial count in samples after use of LISTERINE was 172 with a standard deviation of 8.5 and standard error of 4.9.

### EXAMPLE 6

### Antibacterial Effect of Mouthwash #3

The mouthwash #3 (MW3) is shown in Table 6. This mouthwash was then passed through 0.22 micron sterilizing membrane to remove any bacteria and viruses.

TABLE 6

| Components of MW#3 | Amount in 1000 mL of water |
|---|---|
| Apple cider vinegar | 50 mL |
| Citric acid | 5 mL |
| Mango leaves extract | 5 mL |
| Hibiscus extract | 200 mL (5 grams 100 mL water, 20 min) |
| Green Tea extract | 5 mL (5 grams of dry green tea leaf |
| | extracted with 100 mL of water) |
| 1M Sodium Bicarbonate | 8-10 mL |

### EXAMPLE 7

### Antibacterial Effect of Mouthwash #4

The mouthwash #4 (MW4) is shown in Table 7. This mouthwash was then passed through 0.22 micron sterilizing membrane to remove any bacteria and viruses.

TABLE 7

| Components of MW#3 | Amount in 1000 mL of water |
|---|---|
| Apple cider vinegar | 50 mL |
| Citric acid | 5 mL |
| Mango leaves extract | 10 mL (5 leaves cut into pieces and |
| | extracted with H₂O) |
| Hibiscus extract | 200 mL (2 tbags in 200 mL hotwater, 20 min) |
| 1M Sodium Bicarbonate | 8-10 mL |

### EXAMPLE 8

### Preparation of Toothbrush Cleaning Solution

The toothbrush cleaning solution, hereinafter referred to as tooth brush wash #1 (TBW1) was prepared by mixing 5% acetic acid, and citric acid as lime extract in water. The required amounts of the components in water are shown in Table 8.

TABLE 8

| Components of TBW1 | Amount in 10 mL of water |
|---|---|
| Acetic acid | 1 mL |
| Citric acid | 0.1 mL |

### EXAMPLE 9

### Antibacterial Effect of Toothbrush Cleaning Solution

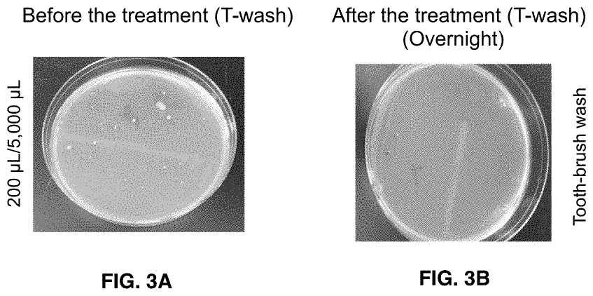

The toothbrush was washed after brushing and was either sprayed with control water (FIG. 3A) or with the toothbrush wash #1 (TBW1, FIG. 3B) for 3 sprays and left overnight (12 hours). The next morning before using, the brush was soaked with shaking in 5 mL of water and this water was tested for bacterial colonies (FIGS. 3A-3B). Table 9 shows the bacterial count in 200 μl/5 ml of toothbrush soaked wash.

TABLE 9

| | | Bacterial count after |
|---|---|---|
| SN | Bacterial count in Control | treatment with TBW1 |
| 1 | 31 | 2 |
| 2 | 23 | 8 |
| 3 | 43 | 7 |

### P Value and Statistical Significance

The two tailed P value equals 0.0120. By conventional criteria, this difference is considered to be extremely statistically significant. Average bacterial count in control was 32 with a standard deviation of 10 and standard error of 5.8. Average bacterial count in samples after use of toothbrush wash #1(TBW1) was 5.6 with a standard deviation of 3.2 and standard error of 1.8.

### EXAMPLE 10

### Preparation of Make-Up Brush Cleaning Solution:

The make-up brush cleaning solution, hereinafter referred to as make-up brush wash #1 (MBW1) was prepared by mixing the 5% acetic acid, 100% propionic acid, and citric acid as lime extract in water. The required amounts of the components in water are shown in Table 10.

TABLE 10

| Components of MBW#1 | mL |
|---|---|
| Acetic acid | 1 mL |
| Citric acid as lime extract | 5 mL |
| Neem extract | 25 μL |

### EXAMPLE 11

### Antibacterial Effect of Make-Up Brush Cleaning Solution

The makeup brush was washed and was either sprayed with control water or with the make-up brush-wash 1 (MBW1) (3 sprays) and left overnight (12 hours). Next day morning before using, the brush was soaked with shaking in 5 mLs of water and this water was tested for bacterial colonies. Table 11 shows the bacterial count in 200 μl/5 ml of make-up brush soaked wash.

TABLE 11

| SN | Bacterial count in control | Bacterial count in MBW1 |
|---|---|---|
| 1 | 23 | 11 |
| 2 | 33 | 9 |
| 3 | 47 | 7 |

### P Value and Statistical Significance

The two tailed P value equals 0.0229. By conventional criteria, this difference is considered to be extremely statistically significant. Average bacterial count in control was 34 with a standard deviation of 12 and standard error of 7. Average bacterial count in samples after use of Make-up brush wash 1 (MBW1) was 9 with a standard deviation of 2 and standard error of 1.1.

### EXAMPLE 12

### Preparation of Toothpaste

The toothpaste composition was prepared by mixing the Balsamic vinegar, mango leaves extract, sodium bicarbonate, xylitol, peppermint, ginger extract, virgin olive oil and coconut oil in water. The required amounts of components in water are shown in Table 12.

TABLE 12
Components of toothpaste

| composition | Amount in 100 mL water |
|---|---|
| Balsamic Vinegar | 1 mL |
| Mango leaves extract | 1 mL |
| Sodium bicarbonate | 10 g |
| Xylitol | 2 g |
| Peppermint | 250 μL |
| Ginger extract | 250 μL |
| Virgin Olive oil | 5 mL |
| Coconut oil | 25 mL |

### EXAMPLE 13

### Preparation of Breath Freshener

The Breath freshener composition was prepared by mixing the balsamic vinegar, mango leaves extract, sodium bicarbonate, xylitol, peppermint, ginger extract in water. The required amounts of components in water are shown in Table 13.

TABLE 13

| Components Breath | |
|---|---|
| freshner composition | Amount in 100 mL water |
| Balsamic Vinegar | 1 mL |
| Mango leaves extract | 1 mL |
| Sodium bicarbonate | 10 g |
| Xylitol | 2 g |
| Peppermint | 250 μL |
| Ginger extract | 250 μL |

### EXAMPLE 14

### Preparation of Chewing Gum

A chewing gum may be prepared with mango leaves extract, sodium bicarbonate, xylitol, peppermint, ginger extract, for example as shown in Table 14.

TABLE 14
Components of chewing

| gum formulation | Amount in 100 g of gum |
|---|---|
| Mango leaves extract | 1 mL |
| Sodium bicarbonate | 10 g |
| Xylitol | 2 g |
| Peppermint | 250 μL |
| Ginger extract | 250 μL |

### EXAMPLE 15

### Preparation of a Cleaner

A cleaner was prepared by mixing the 5% acetic acid, 100% propionic acid, 100% citric acid, rosemary extract, and lemon grass extract in water. The required amounts are shown in Table 13.

TABLE 15

| Components of cleaning | Amount used in 50 mL of |
|---|---|
| composition | water |
| Acetic acid | 12.5 mL |
| Citric acid | 2 mL |
| Propionic acid | 2 mL |
| Rosemary extract | 0.25 mL |
| Lemon grass extract | 0.25 mL |

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. An alcohol-free anti-bacterial cleaner, comprising:
water;
vinegar in an amount of about 10% v/v to about 25% v/v; and
at least one organic acid in an amount of about 1% v/v to about 5% v/v.

2. The alcohol-free anti-bacterial cleaner of claim 1, further comprising at least one natural extract in an amount of about 0.25% v/v to about 20% v/v.

3. The alcohol-free anti-bacterial cleaner of claim 2, wherein the natural extract is neem extract, ginger extract, rosemary, lemon grass extract, mint, peppermint, mango leaves extract, lime extract, hibiscus flower extract, green tea leaf extract, or fennel seed extract, or a combination thereof.

4. The alcohol-free anti-bacterial cleaner of claim 1, wherein said organic acid is citric acid or propionic acid or a combination thereof.

5. The alcohol-free anti-bacterial cleaner of claim 1, wherein said vinegar is 5% acetic acid, 10% acetic acid, balsamic vinegar, or apple cider vinegar.

6. The alcohol-free anti-bacterial cleaner of claim 1, wherein said vinegar is an amount of about 10% v/v.

7. The alcohol-free anti-bacterial cleaner of claim 1, wherein said vinegar is an amount of about 25% v/v.

8. The alcohol-free anti-bacterial cleaner of claim 1, wherein said organic acid in an amount of about 1% v/v.

9. The alcohol-free anti-bacterial cleaner of claim 1, wherein said organic acid in an amount of about 4% v/v.

10. The alcohol-free anti-bacterial cleaner of claim 1, wherein the alcohol-free anti-bacterial cleaner is effective to clean a hard surface, a toothbrush, a brush or a make-up brush.

11. An alcohol-free anti-bacterial hard surface cleaner comprising
water;
vinegar in an amount of about 25% v/v;
at least two organic acids each in an amount of about 4% v/v; and
at least one natural extract in an amount of about 0.5% v/v.

12. The alcohol-free anti-bacterial hard surface cleaner of claim 11, wherein the vinegar is 5% acetic acid, 10% acetic acid, balsamic vinegar, or apple cider vinegar.

13. The alcohol-free anti-bacterial hard surface cleaner of claim 11, wherein said organic acids are citric acid and propionic acid.

14. The alcohol-free anti-bacterial hard surface cleaner of claim 11, wherein said natural extract is neem extract, ginger extract, rosemary, lemon grass extract, mint, peppermint, mango leaves extract, lime extract, Hibiscus flower extract or fennel seed extract, or a combination thereof.

15. The alcohol-free anti-bacterial hard surface cleaner of claim 11, wherein said hard surface is a floor, a wall, a tile, a window, glass, counters, dishes, appliances, fabrics, clothes or carpets.

16. An alcohol-free anti-bacterial brush cleaner comprising:
water;
vinegar in an amount from about 10%;
at least one organic acids in an amount from about 1% v/v to about 5% v/v; and
at least one natural extract in an amount of about 0.25% v/v.

17. The alcohol-free anti-bacterial brush cleaner of claim 16, wherein the vinegar is 5% acetic acid, 10% acetic acid, Balsamic vinegar or Apple cider vinegar.

18. The alcohol-free anti-bacterial brush cleaner of claim 16, wherein the organic acid is citric acid, propionic acid or a combination thereof.

19. The alcohol-free anti-bacterial brush cleaner of claim 16, wherein the natural extract is neem extract, ginger extract, rosemary, lemon grass extract, mint, peppermint, mango leaves extract, Hibiscus flower extract or fennel seed extract, or a combination thereof.

20. The alcohol-free anti-bacterial brush cleaner of claim 16, wherein the brush is a hair brush, a tooth brush or a make-up brush.
